(19) United States
(12) Patent Application Publication
(10) Pub. No.: US 2010/0160913 A1
(43) Pub. Date: Jun. 24, 2010

(54) Surgical Device

(76) Inventor: Enzo Scaglia, Milano (IT)

Correspondence Address: MANNAVA & KANG, P.C., 11240 WAPLES MILL ROAD, Suite 300, FAIRFAX, VA, 22030, US

(21) Appl. No.: 12/602,374

(22) PCT Filed: May 30, 2008
(86) PCT No.: PCT/IB08/01390

§ 371 Date: Nov. 30, 2009

(30) Foreign Application Priority Data
May 31, 2007 (IT) UD2007A000099

(51) Int. Cl. A61B 17/00; A61B 17/66; A61B 17/56

(52) U.S. Cl. 606/57; 606/282; 606/96

(57) ABSTRACT

A surgical device to insert and attach, inside a bone of a limb, an oblong prosthesis provided, in predefined positions, with transverse attachment holes, comprises a support element able to be temporarily coupled with the oblong prosthesis in order to insert the oblong prosthesis into the bone, and a holing template able to be selectively coupled in a predetermined position with the support element, outside the limb. The holing template is coherent with the oblong prosthesis and is able to reproduce at least the predefined position of one or more of the transverse attachment holes.

### FIELD OF THE INVENTION

The present invention concerns a surgical device able to be used during a surgical intervention to reconstruct a fractured bone, particularly but not exclusively the bone of a lower limb of the human body, such as the femur or tibia, by implanting an intra-medullary prosthesis.

### BACKGROUND OF THE INVENTION

It is known that, in a surgical operation, if a bone of a lower limb such as the tibia or femur is broken, the surgeon inserts an intra-medullary prosthesis, of the appropriate shape and size, into the bone. The prosthesis has transverse holes at predefined positions, so as to be attached by screws to the bone itself. The intra-medullary prosthesis is inserted with the aid of a suitable support element coupled with the prosthesis. Afterwards, the surgeon perforates the limb and the bone so as to make passage holes, aligned with the attachment holes of the prosthesis, through which the attachment screws and the possible positioning rods are inserted.

Once the prosthesis has been inserted into the limb, the position of the prosthesis, and particularly that of the transverse attachment holes, is no longer visible from the outside, and this makes it difficult to perforate the bone in exact alignment with said transverse holes.

To obviate this, after having inserted the prosthesis into the bone, an X-ray is usually taken of the limb. The X-ray indicates the internal position of the attachment holes. This has the disadvantage of subjecting the patient, and especially the surgeon, to high doses of radiations, which are harmful over time.

Alternatively, it is known to make the support element which is used to insert the prosthesis into the bone, with a fixed holing mask, in a single piece with the support element. This solution, however, has limited operativeness, it may not be very precise and it is bulky during the step when the prosthesis is inserted.

Purpose of the present invention is to achieve a surgical device which allows the surgeon to have a precise indication of the position of the prosthesis and the relative attachment holes even when the prosthesis is inserted into the bone and therefore is no longer visible to the naked eye and which therefore allows to make the attachment holes through the bone exactly aligned with the relative attachment holes of the prosthesis inserted in the bone, which is not bulky, particularly during the operation to insert the prosthesis itself, and which is applicable to various operating conditions.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

In accordance with the above purpose, a surgical device according to the present invention can be used to insert and fix, in the bone of a limb, an oblong prosthesis provided, in pre-defined positions, with transverse attachment holes, to attach the prosthesis to the bone. The surgical device comprises a support element able to be temporarily coupled with the oblong prosthesis to insert the latter into the bone.

According to a characteristic feature of the present invention, the support element can be associated with a holing template. The holing template is able to be selectively coupled in a predetermined position with the support element, outside the limb. The holing template is coherent with said oblong prosthesis and is able to reproduce at least the predefined position of one or more of said attachment holes.

The holing template is coherent with the prosthesis at least in the sense that its shape and size are such that, once the template has been mounted on the support element and the prosthesis has been inserted into the bone and is therefore hidden from sight, the template functions as an exact reference, external and hence visible to the surgeon, of the relative transverse attachment holes present in the prosthesis, with respect to the support element.

In particular, there will advantageously be guide means for a tool to perforate the bone, by means of which the holing template is able to reproduce the predefined position of one or more of said attachment holes.

Once coupled with the prosthesis, the support element keeps its position constant with respect to the prosthesis. Therefore, it is very easy to determine the precise reciprocal position of the prosthesis, and in particular its attachment holes, and the external template with respect to said support element.

The invention therefore allows to make the passage holes through the bone in exact alignment with the relative attachment holes of the prosthesis inserted into the bone.

Moreover, since the holing template is selectively detachable from the support element coupled with the prosthesis, the invention is not at all bulky, particularly during the operation to insert the prosthesis.

Furthermore, a variant of the present invention allows a plurality of adjustments of the template, so that it can be applied to various operating conditions and various types of prosthesis.

In particular, according to one form of embodiment of the present invention, the holing template comprises guide or reference elements, for example but not only, which can be configured as guide tubes transverse to the bone to be perforated.

Each guide element is able to assume, with respect to the other guide elements, and also with respect to the bone to be perforated, a selectively adjustable position, on each occasion, coordinated with the position of said transverse attachment holes, to guide the tip of a perforating tool so as to make passage holes in the bone.

Alternatively, the guide or reference elements are disposed fixed with respect to each other, in correlation with the predetermined position of the transverse attachment holes.

An advantageous variant provides that the displacement of the guide or reference elements toward the bone to be perforated is effected by using an oblong insert, like an elongated nail, removably located in an external tube that constitutes each of the guide or reference elements.

The oblong insert has a pointed end, or in any case shaped in a tapered manner, which protrudes through an open end of the external tube by a determinate amount. The particular shaping of the tip allows a less invasive insertion through the muscles of the limb.

The pointed end is thus able to be inserted through the muscles of the limb toward the surface of the bone to be perforated.

Once the guide or reference elements have been positioned in the desired position, without damaging the muscles, the oblong insert is removed and in its stead a tip to perforate the bone is inserted.

Advantageously, moreover, the present invention is not harmful for the health of the surgeon or the patient.

### BRIEF DESCRIPTION OF THE DRAWINGS

These and other characteristics of the present invention will become apparent from the following description of a preferential form of embodiment, given as a non-restrictive example with reference to the attached drawings wherein:

FIG. 1 is a front view of part of a surgical device according to the present invention;

FIG. 2 is a front view of another part of a surgical device according to the present invention;

FIG. 3 is a view of a prosthesis inserted into a femur;

FIG. 4 is an enlarged partial section of a part of the surgical device in FIG. 2;

FIG. 5 is an enlarged partial section of another part of the surgical device in FIG. 1;

FIG. 6 is an enlarged partial section of another part of the surgical device in FIG. 1;

FIG. 7 is a part lateral view of a variant of the surgical device in FIG. 1;

FIG. 8 is a section of the variant in FIG. 7;

FIG. 9 is a section of a variant of part of the surgical device in FIG. 1;

FIG. 10 is a section of a component of FIG. 9;

FIG. 11 is a section of another component of FIG. 9.

### DETAILED DESCRIPTION OF A PREFERENTIAL FORM OF EMBODIMENT

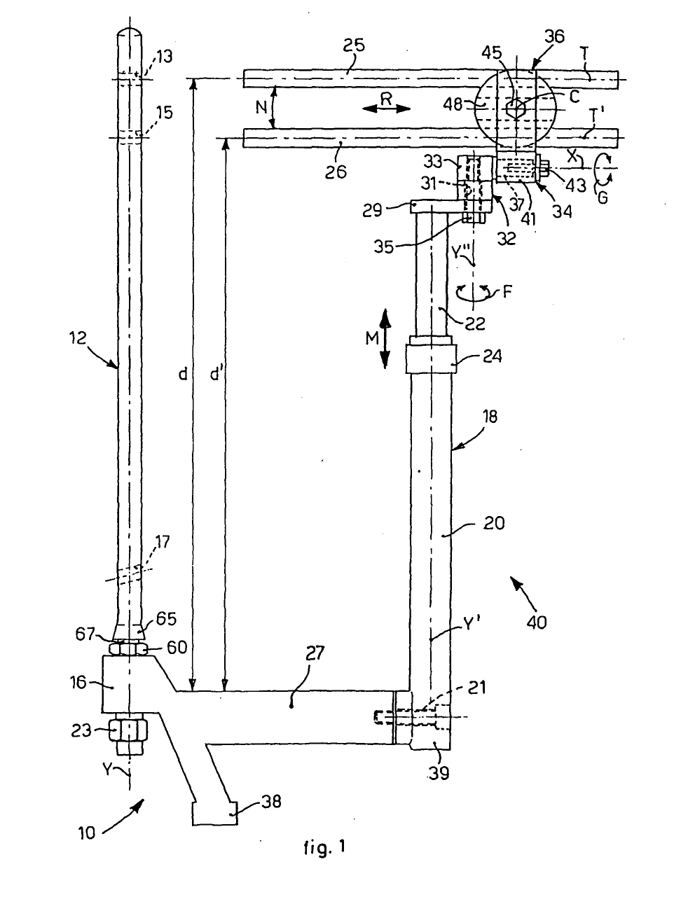

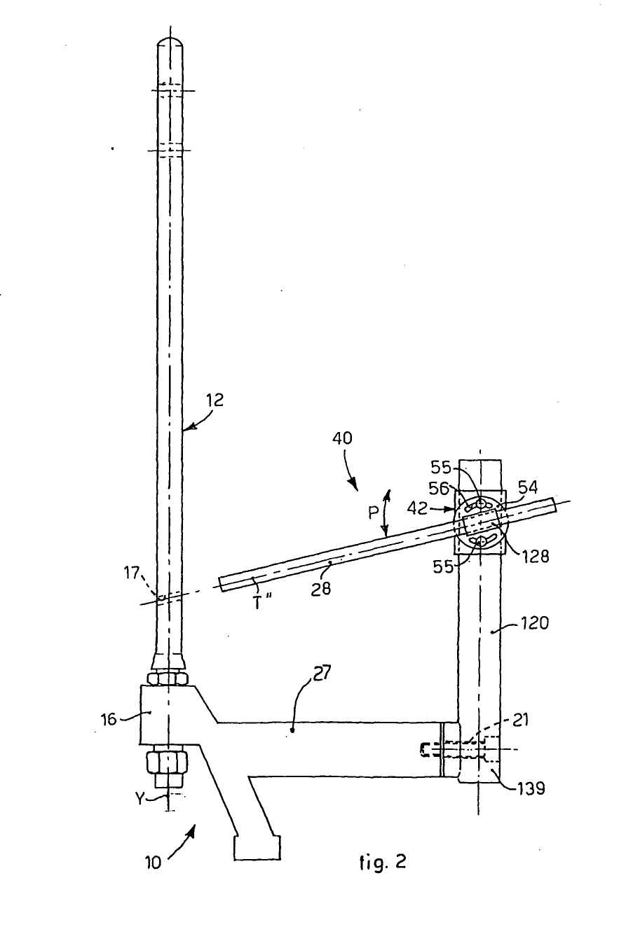

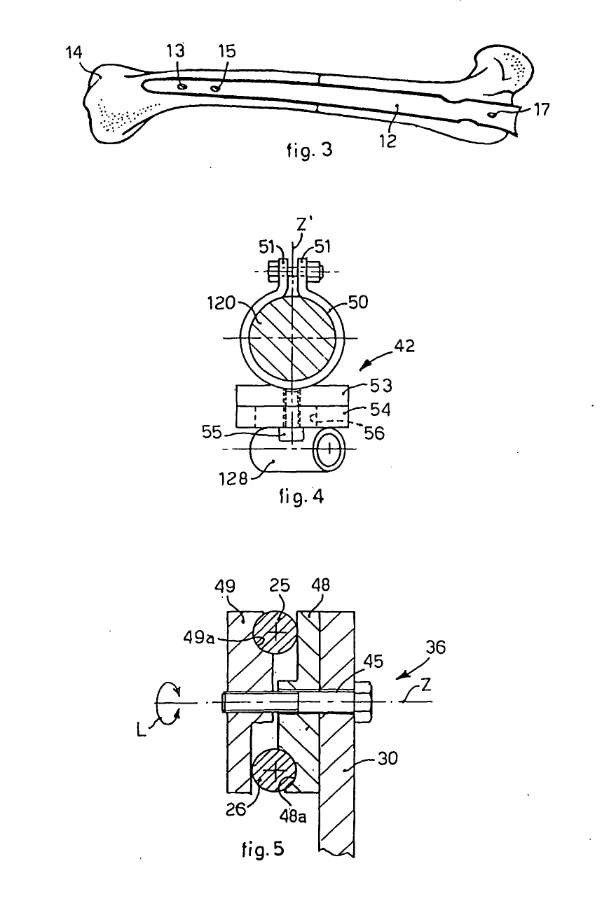

With reference to FIG. 1, a surgical device 10 according to the present invention comprises, in a traditional manner, a support 16 able to be coupled by means of a screw 23 and as will be explained in detail hereafter in the description, with an oblong prosthesis 12, of the intra-medullary type, insertable in a femur 14 (FIG. 3). The support 16 is used to insert the prosthesis 12 into the femur 14 and in particular, is provided with a handle 27, so that the surgeon can grip it, and an abutment portion 38, which the surgeon taps with a suitable hammer specific to the surgical orthopedic field, not shown in the drawings, to insert by force the prosthesis 12 into the femur 14.

The prosthesis 12 is provided with transverse holes 13, 15, 17, a pair of upper holes 13, 15, and a single lower hole 17, by means of which and thanks to attachment screws not shown in the drawings, the prosthesis 12 can be attached stably to the femur 14. To do this, the surgeon has to make passage holes in the femur 14 (FIG. 3) for said screws to pass, in a position correctly aligned with said holes 13, 15, 17, by means of the tip of a suitable drill, specific to the surgical orthopedic field, not shown in the drawings.

In order to make the passage holes correctly in the femur 14, corresponding to the holes 13, 15 and 17, the device 10 comprises a holing template 40 (FIGS. 1 and 2).

The size and/or shape of the template 40 are coherent with the prosthesis to be inserted, so as to be able to function as an exact external reference for the surgeon, when the prosthesis 12 is inserted into the femur 14 and hence no longer visible.

The template 40 comprises three tubular guide elements 25, 26 and 28, of a length compatible with the extension of the support 16.

The corresponding cavities of the three tubes 25, 26 and 28 function as reference guides along the respective longitudinal axes T, T′ and T″, for the perforating action of the drill bit, aligned with said holes 13, 15 and 17.

As can be seen, in ideal perforating conditions, the axes T, T′ and T″ are aligned with the axes of the holes 13, 15 and 17 (FIGS. 1 and 2).

The template 40 has a terminal part 39 (FIG. 1) which can be selectively coupled univocally, by means of a screw 21, with the support 16, so as to support the guide tubes 25 and 26, and another terminal part 139, able to be mounted as an alternative to part 39, also able to be selectively coupled univocally, by means of the screw 21 with the support 16, in order to support the tube 28, as will be shown hereafter.

In this way, before inserting the prosthesis 12 into the femur 14, the surgeon couples the template 40 with the support 16, with either the part 39 or the part 139, and prepares the tubes 25, 26 and 28 in the correct position, in coordination and coherent with the predefined position of each of the holes 13, 15 and 17 of the prosthesis 12.

Following this positioning, the surgeon separates the template 40 from the support 16 and thus inserts the prosthesis 12 into the femur 14, without the bulk and inconvenience of performing said operation with the template 40 connected to the prosthesis 12.

Once the prosthesis 12 has been inserted, the surgeon again couples the template 40 to the support 16, thus reproducing the correct conditions of alignment and the correct reference position of the holes 13, 15 and 17 of the prosthesis 12, thanks to the univocal coupling template 40 —support 16 and the correct positioning of the tubes 25, 26 and 28 made previously. Advantageously, moreover, during this step the guide tubes 25, 26 and 28 are made to slide along the relative axes T, T′ and T″ and are thus able to be positioned with the respective ends in determinate positions, in substantial contact, or in any case in close proximity, with the femur 14. In this way the tip of the drill, guided inside the tubes 25, 26 and 28, is prevented from slipping or inclining with respect to the longitudinal axis of the latter, for example due to irregularities in the surface of the bone or to stresses occurring during the drilling operation.

In this way the surgeon, although he cannot actually see where the holes 13, 15 and 17 of the prosthesis 12 are, is able to understand their disposition thanks to the correct alignment referred by the tubes 25, 26 and 28. At this point, the surgeon can correctly carry out the drilling by means of the drill bit, through the tubes 25, 26 and 28.

Alternatively, the template 40 can remain coupled with the prosthesis 12 while the latter is inserted into the femur 14. The tubes 25, 26 or 28 must be suitably distanced from the prosthesis 12 by making them slide along the respective longitudinal axes T and T′, so as to make the patient's limb pass in the space between the prosthesis 12 and the template 40.

Advantageously the template 40 comprises an adjustment unit, formed by adjustment members 18, 32, 34, 36 and 42, which allows a wide range of spatial adjustments of the tubes 25, 26 and 28, so as to easily adapt to a wide range of types of prosthesis 12, both in shape and in size, and also in the position of the holes in the prosthesis.

To give a non-restrictive example, we shall now illustrate some forms of embodiment of the adjustment members 18, 32, 34, 36 and 42. It is clear that, according to needs, the functions of two or more of these adjustment members can be grouped together in a single adjustment device.

In particular, the adjustment unit comprises a telescopic member 18 which allows a linear adjustment in height of the position of the tubes 25 and 26, along an axis Y′ parallel to the longitudinal axis Y of the prosthesis 12 when the prosthesis 12 is coupled with the template 40, as shown by the arrow M.

The extension in height of the telescopic member 18 is directly correlated to the longitudinal dimension of the prosthesis 12, so as to be able to adjust the distance d and d′, respectively of the tube 25 and 26, from the support 16. In particular, the telescopic member 18 is formed by two uprights 20 and 22 coaxial with the axis Y′, of which an external upright 20 attached to the terminal part 39 of the template 40 and a telescopic internal upright 22, sliding in the upright 20. The relative position of the two uprights 20 and 22 is selectively determined by means of a clamping ring-nut 24, easily maneuverable from the outside by the surgeon or by a suitable operator.

Three adjustment members 32, 34 and 36 in particular allow the angular adjustment of the two tubes 25 and 26, and an adjustment member 42 allows the linear and angular adjustment of the tube 28, according to contingent operating necessities.

The adjustment member 32 allows to rotate and selectively clamp in a determinate angular position the tubes 25 and 26 with respect to an axis Y″, parallel to the axes Y and Y′ mentioned above, as indicated by the arrow F. To this purpose, the member 32 comprises a horizontal plate 29, attached to the upper end of the upright 22 on which, by means of a vertical pin 31, a supporting block 33 is pivoted around the axis Y″. The angular position of the block 33 can be selectively fixed, by means of a clamping ring-nut 35 of the vertical pin 31, and this, as is clear from the attached drawings and from the following description, also determines the angular position of the tubes 25 and 26 around the axis Y″.

The adjustment member 34 allows to rotate and selectively clamp in a determinate angular position the tubes 25 and 26 with respect to an axis X, orthogonal and co-planar to the axes Y and Y′ mentioned above, as indicated by the arrow G. The member 34 consists of another block 41 which by means of a horizontal pin 37 is pivoted to said block 33 around the axis X. The angular position of the block 41 can be selectively fixed, by means of a clamping nut 43 of the vertical pin 37, and this, as is clear from the attached drawings, also determines the angular position of the tubes 25 and 26 around the axis X.

The adjustment member 36 allows to rotate and selectively clamp in a determinate angular position the tubes 25 and 26 with respect to an axis Z (FIG. 5), orthogonal to the plane defined by the axes X, Y′ and Y″ and identified in FIG. 1 by the rotation point or center C.

In particular, the member 36 comprises a plate 30, mounted on said block 41, and on which two circular plates 48 and 49 are mounted, each having a housing seating 48a, 49a in which one end of the tubes 25 and 26 is positioned. The plates 48 and 49 are pivoted in correspondence with point C by means of a screw 45.

The plates 48 and 49 can be selectively clamped in a single piece, by means of the screw 45, so as to be rotated simultaneously around said axis Z and thus determine the angular position of both tubes 25 and 26 around said axis Z, as indicated by the arrow L in FIG. 5.

Moreover, the adjustment member 36 also allows to adjust the reciprocal angular position of the tubes 25 and 26 with respect to the center C, substantially scissor-wise, allowing to spread them and bring them together, as indicated by the arrow N in FIG. 1.

To do this, the screw 45 is released, the plates 48 and 49 are rotated independently of each other and again clamped by means of the screw 45, in determinate angular positions.

By means of the adjustment member 36, moreover, it is possible to displace the tubes 25 and 26 linearly along the respective longitudinal axes T and T′, bringing them closer to, and distancing them from, the axis Y of the prosthesis 12, in desired positions, as indicated by the arrow R in FIG. 1. It is sufficient to release the screw 45 and make the tubes 25 and 26 slide in the seatings 48a and 49a along the axes T and T′, for the desired length, and again clamp everything with the screw 45. This is advantageous, as we said, because it allows to position the tubes 25 and 26 in contact with the femur 14 so that, during drilling, the drill bit does not slide or incline with respect to the longitudinal axis T and T′.

Finally, the adjustment member 42 is mounted on the part 139, in particular on a vertical upright 120 of the latter. The member 42 allows to adjust the tube 28 both linearly along the axis Y′, and also angularly with respect to an axis Z′, orthogonal to the axis Y′. To this purpose, the member 42 (FIG. 4) comprises a bushing 50, able to be fitted on the upright 120 and attached thereto by means of two fins 51 clamped by a screw 52. The bushing 50 is thus positionable along the axis Y′ at a desired height.

A supporting plate 53 is mounted on the bushing 50, fixed with respect to the bushing 50, and a circular plate 54 provided with two annular eyelets 56, concentric with respect to the axis Z′. In turn, the plate 54 solidly supports the bushing 128, in which the tube 28 is inserted and which allows it to slide along the axis T″ for positioning in substantial proximity with the femur 14. Everything is attached to the bushing 50 by means of two screws 55, which engage in said annular eyelets 56 of the plate 54.

The plate 54 thus has the possibility of rotating guided by the annular eyelets 56 as indicated by the arrow P, and positioned in a desired angular position, determining the desired inclination of the bushing 128 and therefore of the guide tube 28.

Furthermore, since the bushing 50 can slide along the upright 20, the vertical position of the bushing 128, and hence of the tube 28, is thus adjustable in height.

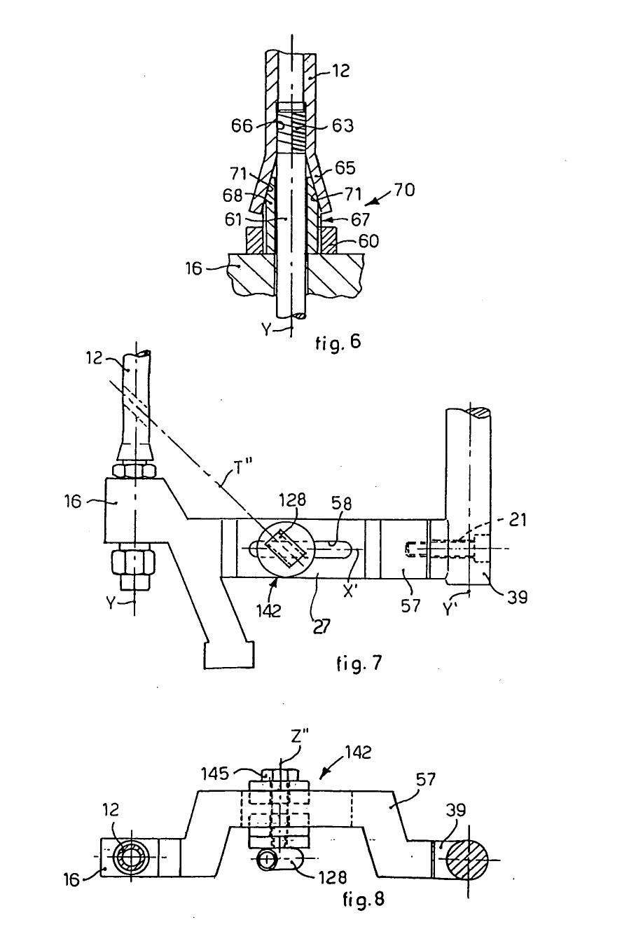

According to a variant of said adjustment member 42, indicated for convenience by the reference number 142, shown in FIGS. 7 and 8, where the same reference numbers correspond to equal parts, the tube 28 is mounted on a bar 57, made in association with or as an alternative to the handle 27, which connects the terminal 39 of the template 40 with the support 16 for the prosthesis 12. In this case the adjustment member 142 has a screw 145 which positions it on the arm 57. The screw 145 is inserted into a linear eyelet 58 of the arm 57, so that the member 142 as a whole is able to slide in order to adjust the position of the bushing 128, and hence of the tube 28, along an axis X′, orthogonal to the axis Y of the prosthesis 12, so as to selectively bring closer or distance the tube 28 with respect to the prosthesis 12. Moreover, the tube 28, thanks to the screw 145, is also able to be selectively oriented in determinate angular positions around an axis Z″, orthogonal to said axis X′. In this variant, the arm 57 is advantageously shaped with a recess, as can be seen in FIG. 8, in which the adjustment member 142 is housed; the recess is of suitable size, so as to allow optimum alignment between tube 28 and hole 17 of the prosthesis 12.

It should be noted that the coupling of the support 16 and the prosthesis 12 is important for the present invention, since the constancy and security of the position of the support 16 with respect to the prosthesis 12 is the basis for the exact alignment allowed by the template 40. In other words, the support 16 is a fixed and unaltered reference for the surgeon, between the condition in which the prosthesis 12 is outside the limb and the template 40 is coupled and adjusted in coherence with the prosthesis 12, and the condition in which the prosthesis 12 is in the femur 14, hidden from view, and the template 40 is again coupled thereto in order to make the passage holes in the femur 14.

Moreover, this coupling between the support 16 and the prosthesis 12 must prevent not only axial movements but also unwanted rotations of the prosthesis 12 around its axis Y, because if that were to happen there would be an angular misalignment of the position of the holes 13, 15 and 17 and the corresponding tubes 25, 26 and 28. In this case, the holes 13, 15 and 17 would be at a height coordinated with the height of the tubes 25, 26 and 28, but rotated and hence out of phase, and it would be impossible to align and insert the attachment screws of the femur 14.

To this purpose, and according to an advantageous form of embodiment of the present invention, the support 16 comprises an innovative attachment unit 70 for coupling with the prosthesis 12 (FIG. 6). The attachment unit 70 comprises a tubular element 61 with a threaded end 63. The prosthesis 12 has a flared or bell-shaped end 65, able to be fitted on the tube 61 and, inside, it is shaped as a female thread 66 mating with the threaded end 63 of the tube 61. A bushing 67 is able to be fitted on the tube 61, and during use is inserted into the flared end 65 of the prosthesis 12, between the tube 61 and the prosthesis 12. In particular, the bushing 67 has a conical portion able to be inserted into said end 65, and also an external threaded part 69, of a cylindrical shape coordinated with the section of the tube 61, on which a corresponding nut 60 is screwed, internally threaded in a manner mating with the bushing 67. The conical portion 68 advantageously has a not completely curvilinear cross section, that is, with at least an anti-rotation rectilinear segment 71, advantageously two, which, cooperating with the internal surface of the flared end 65, prevent the rotation of the prosthesis 12 around its axis Y.

In this way an effective axial and angular clamping is achieved of the support 16 and the prosthesis 12, which are therefore joined as in a single piece, and also a effective unclamping thereof.

The coupling and clamping is achieved by inserting the prosthesis 12 on the bushing 67 and screwing the tube 61, by means of the screw 23. Furthermore, the conical coupling with the conical part 68 of the bushing 67, following the surgeon's action with the hammer on the abutment portion 38, is made still more solid and difficult to release. In the state of the art this release is usually achieved by releasing the screw 23 and by hitting the end 65 of the prosthesis lightly and repeatedly, in order to uncouple it from the conical part 68. This can obviously lead to damage both to the femur 14 and also to the prosthesis 12.

Advantageously, on the contrary, the present invention allows to release the prosthesis 12 from the conical part 68 safely and quickly. In fact, according to the present invention, once the screw 23 has been unscrewed, the nut 60 is screwed toward the end 65 of the prosthesis 12 until it abuts thereon, releasing the coupling with the conical part 68. This release is quick and easy, since it effectively exploits the rotary torque imparted to the nut, transforming it into axial thrust on the prosthesis 12.

The attachment unit 70 is also advantageous because it allows to rapidly adapt the device 10 to the different conformation and sizes of the prosthesis 12, in particular the end 65, normally used in surgical practice, without changing the whole support 16. Indeed it is sufficient to change the type of tube 61 and bushing 67, selecting them according to requirements.

According to a variant, instead of making three different adjustment members 32, 34 and 36, each of which defining a corresponding axial, vertical or horizontal pivoting, and hence a single possibility of adjustment along a relative axis, it is possible to integrate the adjustment members into a single adjustment member, using a ball joint which allows to orient the tubes 25 and 26 in space according to needs.

Another modification is to make the template 40 usable only in correlation with a specific prosthesis 12 of determinate sizes and positions of the holes. In this variant the template 40 has the guide tubes 25, 26 and 28 disposed fixed on three corresponding vertical uprights, in a predetermined fixed position corresponding to the position of the holes of the specific prosthesis 12. This solution has the advantage that it is not necessary to make telescopic elements or other adjustment members.

Moreover, a person of skill can replace the guide tubes for the drill bit with other guide devices, of a mechanical, electronic or optical type or a combination thereof, in an equivalent manner.

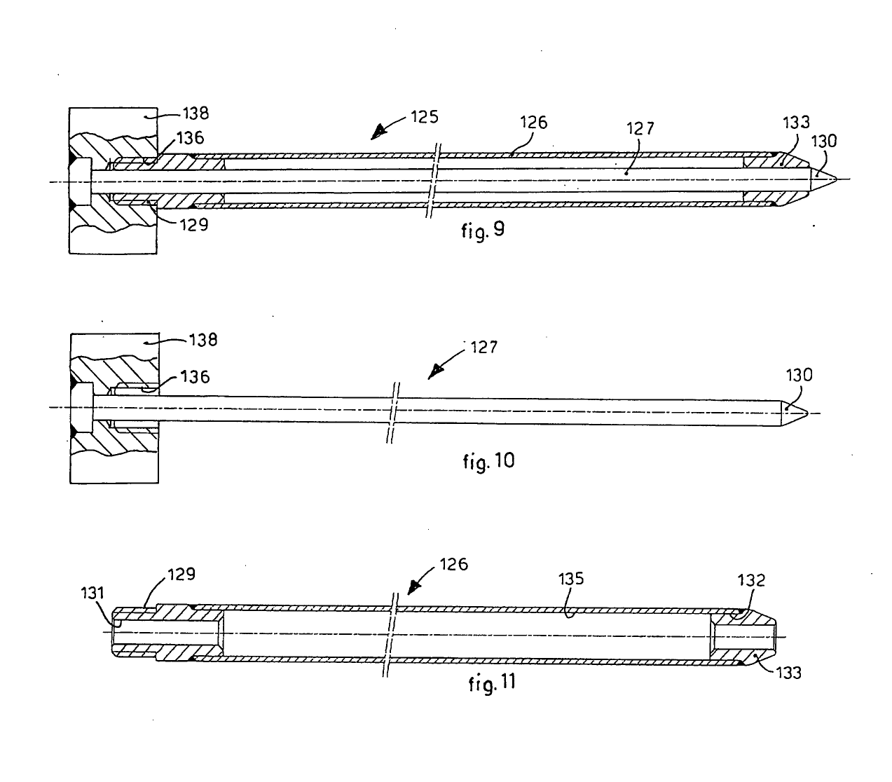

FIGS. 9,10 and 11 show a variant, indicated for convenience by the reference number 125, of one of the guide tubes 25, 26, 28, which is based on the concept of providing an oblong insert 127 which functions as an insertion element through the muscles of the limb, but which, thanks to its shape, does not ruin the muscles during this operation and which, once it has served its purpose, can be removed.

This variant guide tube 125 comprises an external guide 126, axially holed internally in through manner, so as to define an axial cavity 135. The axial cavity 135 has a first aperture 131, through which the oblong insert 127 is housed inside, shaped like an elongated nail compatible with the section of the external guide 126, and a second aperture 132, able to face the bone that is to be perforated, bearing a tapered bushing 133.

The external surface of the tapered bushing 133 is shaped converging, so as to achieve a lead-in and through which a pointed end 130 of the oblong insert 127 protrudes. The tapered bushing 133 is thus fitted on the pointed end 130, allowing easy insertion into the muscles, thanks to its overall shape.

The oblong insert 127 has a bolted end 138 with threading 136, able to be screwed with a female threaded end 129, made in correspondence with the first aperture 131 of the external guide 126. In this way the oblong insert 127 is made selectively removable from the external guide 126.

The combination of the tapered bushing 133 and the pointed end 130 allows to insert the guide tube 125 in a less invasive manner through the muscles of the limb to be operated on, until the pointed end 130 goes into contact with the bone to be perforated.

Once the guide tube 125 has been positioned in proximity or in contact with the bone 14, the oblong insert 127 can be unscrewed and the drill bit to perforate the bone can be inserted in its stead.

In this way, the guide tube 125 is positioned in correspondence with the bone without damaging the surrounding muscles excessively. Indeed, the pointed shape, defined in cooperation by the tapered bushing 133 and the pointed end 130 that is achieved, allows a less invasive insertion, reducing to a minimum the parts of the muscle that are affected by the operation.

According to a variant solution, not shown in the drawings, it may be provided to make, through the handle 27, a through hole inclined by 45°, but it could also be a different inclination according to needs, towards the center line of the prosthesis 12, in which to house one of the guide tubes 25, 26 28 or 125, so as to guide the drilling tool. This solution is analogous to the solution shown in FIGS. 7 and 8, with regard to the positioning of the drilling tool; however, it differs inasmuch as no variations in inclination are provided, since the through hole has a fixed angular position. At the same time, however, this solution offers advantages of compactness, reduced bulk and less complexity in manufacture.

It is clear that modifications and/or additions of parts may be made to the surgical device as described heretofore, without departing from the field and scope of the present invention. It is also clear that, although the present invention has been described with reference to specific examples, a person of skill in the art shall certainly be able to achieve other equivalent forms of surgical device, having the characteristics as set forth in the claims and hence all coming within the field of protection defined thereby.

1. A surgical device to insert and attach, inside a bone of a limb, an oblong prosthesis provided, in predefined positions, with transverse attachment holes, comprising a support element able to be temporarily coupled with said oblong prosthesis in order to insert said oblong prosthesis into said bone, the surgical device also comprising a holing template able to be selectively coupled in a predetermined position with said support element, outside said limb, wherein said holing template is coherent with said oblong prosthesis and is able to reproduce at least the predefined position of one or more of said transverse attachment holes.

2. The surgical device as in claim 1, wherein said holing template comprises guide or reference elements, each of which is able to assume a selectively adjustable position, on each occasion, in a coordinated manner with the position of said transverse attachment holes, to guide the tip of a drilling tool to achieve passage holes in said bone.

3. The surgical device as in claim 2, wherein said template comprises a plurality of adjustment members able to selectively pre-determine and pre-fix the position of one or more of said guide or reference elements with respect to said attachment holes, so that the resulting position of each of said passage holes is coordinated with the predefined position of each of said attachment holes.

4. The surgical device as in claim 3, wherein a first of said adjustment members is able to selectively determine the distance (d, d′) between at least two first of said guide or reference elements and said support element, along a first direction of linear adjustment (Y′) substantially parallel to the longitudinal axis (Y) of said oblong prosthesis.

5. The surgical device as in claim 3, wherein a second of said adjustment members is able to selectively determine the angular position of the guide or reference elements with respect to a first pivoting axis (Y″) substantially parallel to said first direction of linear adjustment (Y′).

6. The surgical device as in claim 3, wherein a third of said adjustment members is able to selectively determine the angular position of the guide or reference elements with respect to a second pivoting axis (X) substantially orthogonal to said first direction of linear adjustment (Y′).

7. The surgical device as in claim 3 wherein a fourth of said adjustment members is able to selectively determine the angular position of the guide or reference elements around a center of rotation (C) associated with an axis of rotation (Z) orthogonal both to said second pivoting axis (X) and also to said first direction of linear adjustment (Y′).

8. The surgical device as in claim 7, wherein said fourth adjustment member is able to selectively determine the angular position of a first of said two guide or reference elements with respect to a second of said two guide or reference elements around said axis of rotation (Z).

9. The surgical device as in claim 7, wherein the fourth adjustment member is also able to selectively determine the linear position of said two guide or reference elements along the respective longitudinal axes (T, T′), with respect to the longitudinal axis (Y) of said oblong prosthesis.

10. The surgical device as in claim 3, wherein a fifth of said adjustment members is able to selectively determine the position of a third of said guide or reference elements both linearly along said first direction of linear adjustment (Y′) and also angularly around an axis (Z′) orthogonal to said first direction of linear adjustment (Y′).

11. The surgical device as in claim 10, wherein the fifth adjustment member is also able to selectively determine the linear position of the third guide or reference element along the respective longitudinal axis (T″), with respect to the longitudinal axis (Y) of said oblong prosthesis.

12. The surgical device as in claim 3, wherein a fifth of said adjustment members is able to selectively determine the position of a third of said guide or reference elements both linearly along a second direction of linear adjustment (X′) orthogonal to said longitudinal axis (Y) of said oblong prosthesis, and also angularly around an axis (Z″) orthogonal to said second direction of linear adjustment (X′).

13. The surgical device as in claim 1, comprising an attachment unit for the selective coupling of said oblong prosthesis with said support element, which comprises:
a tubular element inserted into said support element and able to be coupled with a threaded end of said oblong prosthesis;
an externally threaded bushing able to be fitted on said tubular element and having a conical part able to be inserted into said threaded end to achieve a conical coupling with said oblong prosthesis; and
a clamping element able to be selectively driven to uncouple said threaded end of said oblong prosthesis from said conical part of said bushing.

14. The surgical device as in claim 1, wherein said holing template comprises guide or reference elements, each of which is disposed in a predetermined fixed position, in a coordinated manner with the predefined position of said transverse attachment holes, to guide the tip of a drilling tool in order to make passage holes in said bone.

15. The surgical device as in claim 2, wherein the guide or reference elements consist of an external tube in which an oblong insert is housed, having a pointed end protruding through an open end of the external tube, by a determinate amount, which is able to be inserted through the muscles of the limb, toward the surface of the bone to be perforated.

16. The surgical device as in claim 15, wherein the open end of the external tube has a tapered element fitted on the pointed end, so as to achieve, all in all, a lead-in for the insertion of the guide element.

17. A method to determine, by means of a surgical device, the correct position of attachment holes of an oblong prosthesis inserted inside a bone of a limb, wherein the oblong prosthesis is able to assume a first condition, in which it the oblong prosthesis is visible, and a second condition in which the oblong prosthesis is inserted inside the bone, the method comprising a first step in which, in the first condition of said oblong prosthesis, a holing template is coupled with said oblong prosthesis in a determinate position, by means of a support element, so as to identify in the holing template at least the coordinated predefined position of one or more of the attachment holes, a second step in which the oblong prosthesis is made to pass from the first condition to the second condition and a third step in which, in the second condition of the oblong prosthesis, the holing template is configured with respect to the oblong prosthesis in the determinate position so as to reproduce the predefined position of one or more of the attachment holes also in the second condition of said oblong prosthesis.

18. The method as in claim 17, wherein, in the second step, the holing template is uncoupled from the oblong prosthesis and in the third step is again coupled with the oblong prosthesis.

19. The method as in claim 17, wherein, in the second step, one or more guide or reference elements of the holing template are distanced from the oblong prosthesis and in the third step said guide or reference elements are again brought close to the oblong prosthesis.

20. The method as in claim 19, wherein the insertion of the guide or reference elements toward the bone to be perforated is effected with the aid of an oblong insert, located in an external tube which constitutes each of the guide or reference elements, said oblong insert having a pointed end protruding through an open end of the external tube by a determinate amount, which is able to be inserted through the muscles of the limb, toward the surface of the bone to be perforated, and wherein, once the guide or reference elements have been positioned in the desired position, the oblong insert is removed and in the stead thereof a drill bit is positioned for the perforation of the bone.
